(19) United States
(12) Patent Application Publication
(10) Pub. No.: US 2024/0285970 A1
(43) Pub. Date: Aug. 29, 2024

(54) APPARATUS AND METHOD FOR INACTIVATING VIRUSES IN HUMAN BODY

(71) Applicant: CHUNGMED CO., LTD., Seoul (KR)

(72) Inventors: Hyun Hoon CHUNG, Seoul (KR); Jongheon KIM, Seoul (KR); Seihoon PARK, Seoul (KR)

(73) Assignee: CHUNGMED CO., LTD., Seoul (KR)

(21) Appl. No.: 18/585,112

(22) Filed: Feb. 23, 2024

(30) Foreign Application Priority Data
Feb. 23, 2023 (KR) 10-2023-0024303
Feb. 23, 2023 (KR) 10-2023-0024308
Apr. 20, 2023 (KR) 10-2023-0051960
Apr. 20, 2023 (KR) 10-2023-0051963

(51) Int. Cl. A61N 5/06; G16H 20/40

(52) U.S. Cl. CPC A61N 5/0624; A61N 5/0601; G16H 20/40; A61N 2005/0611; A61N 2005/0626; A61N 2005/0652; A61N 2005/0661

(57) ABSTRACT

Provided are an apparatus and a method for inactivating viruses in human body, the apparatus comprising a body unit including a light emitting unit and formed to be capable of inserting into the human body; an operating unit coupled to the body unit to control the light emitting unit; and a diffusor disposed on an upper portion of the light emitting unit to diffuse light emitted from the light emitting unit, wherein the light emitting unit includes a substrate having a rectangular polyhedral shape and a plurality of ultraviolet light emitting diodes (LEDs) disposed on a surface of the substrate.

### A. TECHNICAL FIELD

The present disclosure relates to inactivating viruses in human body, more particularly, to an apparatus and method for inactivating human papillomavirus (HPV) in human's body using light.

### B. DESCRIPTION OF THE RELATED ART

In general, cervical cancer is a type of cancer that occurs when cells in the cervix, the lower part of the uterus that connects to the vagina, grow abnormally. It is the second most common cancer in women after breast cancer, and the number of patients and deaths is increasing.

Cervical cancer can be caused by a number of factors, but it is most often caused by the persistent infection of the normal cervical cells with the human papillomavirus (HPV).

There are a number of screening methods for cervical cancer. The most common method is the Pap test, which involves inserting a small tool into the cervix to collect cells and then examining the cells for abnormalities. During this process, HPV infection can be tested through genetic analysis. Another method is to visualize the cervix by magnifying it. In severe cases, CT, MRI, and whole-body PET/CT scans are used to determine if the cancer has spread.

Treatment for cervical cancer varies depending on the stage. In the early stages, surgery is usually performed. In more advanced cases, concurrent chemoradiotherapy is used. In some cases, systemic chemotherapy may be used alone. However, even if the uterus is removed by surgery, HPV can continue to exist in the vagina. This can lead to cervical cell dysplasia, and even cervical cancer. Therefore, the development of a safe and effective treatment that inactivates the virus is urgently needed. Therefore, methods to address these problems are required.

### SUMMARY OF THE DISCLOSURE

In one aspect of the present disclosure, an apparatus for inactivating viruses in human body comprises a body unit including a light emitting unit and formed to be capable of inserting into the human body, an operating unit coupled to the body unit to control the light emitting unit; and a diffusor disposed on an upper portion of the light emitting unit to diffuse light emitted from the light emitting unit, wherein the light emitting unit includes a substrate having a rectangular polyhedral shape and a plurality of ultraviolet light emitting diodes (LEDs) disposed on a surface of the substrate.

Desirably, the diffusor may be coupled with a housing formed along an edge of the substrate.

Desirably, the diffusor may be disposed in a film form on a light transmission part of the body unit.

Desirably, the light emitting unit may further include an image sensor disposed on the substrate, and the image sensor is disposed at a center of the substrate.

Desirably, the operating unit may include an indicator that displays a signal for an user guidance based on information obtained by the image sensor.

Desirably, the operating part may include a button that is electrically connected to the light emitting and controls the light emitting unit, and the button may control at least one of an illuminance of light, a wavelength of light, an emission pattern of light emitted from the plurality of ultraviolet LEDs.

Desirably, the plurality of ultraviolet LEDs may include at least two groups of ultraviolet LEDs with different wavelengths.

In another aspect of the present disclosure, an apparatus for inactivating viruses in human body comprises a body unit that includes a light emitting unit and formed to be capable of inserting into the human body and an operating unit that is coupled to the body unit and includes a controller for electrically controlling the light emitting unit, wherein the light emitting unit includes a substrate, each of an image sensor and a plurality of ultraviolet light emitting diodes (LEDs) disposed on the substrate, and wherein the light emitting unit irradiates ultraviolet light to a cervix based on a cervix image captured by the image sensor.

Desirably, the image sensor may be disposed on a center of the substrate, the plurality of the ultraviolet LEDs may be radially arranged around the image sensor.

Desirably, the controller may include a machine learning model and extract feature information from the cervix image using the machine learning model, and generate a signal for operating an indicator of the operating unit according to the feature information.

Desirably, the feature information may include at least one of a presence, a size, a location of the cervix.

Desirably, the controller may generate a signal for operating an indicator of the operating unit according to a size of the cervix obtained from the cervix image.

Desirably, the controller may generate a signal for operating an indicator of the operating unit by comparing a size of the cervix obtained from the cervix image with a reference size of the cervix.

Desirably, the reference size of the cervix may be set differently depending on age.

In further aspect of the present disclosure, an apparatus for inactivating viruses in human body comprises an image sensor that is inserted into a vagina to take an image of a cervix, a controller that extracts feature information of the image of the cervix using a machine learning model and a light source that irradiates ultraviolet light to the vagina or the cervix based on the feature information.

Desirably, the apparatus may further comprise an indicator operated under control of the controller according to a reference value of the feature information.

Desirably, the feature information may include at least one of a presence, a size, a location of the cervix.

### BRIEF DESCRIPTION OF THE DRAWINGS

FIG. 1 is a schematic diagram of an illustrative apparatus inserted into vagina according to embodiments of the present disclosure.

FIG. 2 is a perspective diagram of an illustrative apparatus for inactivating viruses in a body according to embodiments of the present disclosure.

FIG. 3 is a cross-sectional diagram of a single UV LED package of a light-emitting unit according to embodiments of the present disclosure.

FIG. 4 is a diagram illustrating a portion of the body unit including a light-emitting unit according to a first embodiment of the present disclosure.

FIG. 5 is a diagram illustrating a portion of the body unit including a light-emitting unit according to a second embodiment of the present disclosure.

FIG. 6 is a diagram illustrating a portion of the body unit including a light-emitting unit according to a third embodiment of the present disclosure.

FIG. 7 is a diagram of an apparatus which includes a body unit in FIG. 6 according to embodiments of the present disclosure.

FIG. 8 is a flow diagram illustrating a method for inactivating viruses inside a human body according to an embodiment of the present disclosure.

FIG. 9 is a diagram of another apparatus which includes a body unit in FIG. 6 according to embodiments of the present disclosure.

FIG. 10 is a block diagram explaining a training method of a controller of an apparatus according to embodiments of the present disclosure.

FIG. 11 is a flow diagram illustrating a method for inactivating viruses inside a human body according to another embodiment of the present disclosure.

### DETAILED DESCRIPTION OF PREFERRED EMBODIMENTS

In the following description, for purposes of explanation, specific details are set forth in order to provide an understanding of the disclosure. It will be apparent, however, to one skilled in the art that the disclosure can be practiced without these details.

Furthermore, one skilled in the art will recognize that embodiments of the present disclosure, described below, may be implemented in a variety of ways, such as a process, an apparatus, a system, a device.

Components shown in diagrams are illustrative of exemplary embodiments of the disclosure and are meant to avoid obscuring the disclosure. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components that may be implemented in software, hardware, or a combination thereof.

It shall also be noted that the terms “coupled,” “connected,” “linked,” or “communicatively coupled” shall be understood to include direct connections, indirect connections through one or more intermediary devices.

Furthermore, one skilled in the art shall recognize: (1) that certain steps may optionally be performed; (2) that steps may not be limited to the specific order set forth herein; and (3) that certain steps may be performed in different orders, including being done contemporaneously.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” or “in embodiments” in various places in the specification are not necessarily all referring to the same embodiment or embodiments.

The terms “comprise/include” used throughout the description and the claims and modifications thereof are not intended to exclude other technical features, additions, components, or operations.

Unless the context clearly indicates otherwise, the singular forms “a,” “an,” and “the” are intended to include the plural forms as well. Also, when description related to a known configuration or function is deemed to render the present disclosure ambiguous, the corresponding description is omitted.

In the following description, it shall also be noted that the terms “learning” shall be understood not to intend mental action such as human educational activity because of referring to performing machine learning by a processing module such as a processor, a CPU, an application processor, micro-controller, so on.

An “image” is defined as a reproduction or imitation of the form of a person or thing, or specific characteristics thereof, in digital form. An image can be, but is not limited to, a JPEG image, a PNG image, a GIF image, a TIFF image, or any other digital image format known in the art. “Image” is used interchangeably with “photograph”.

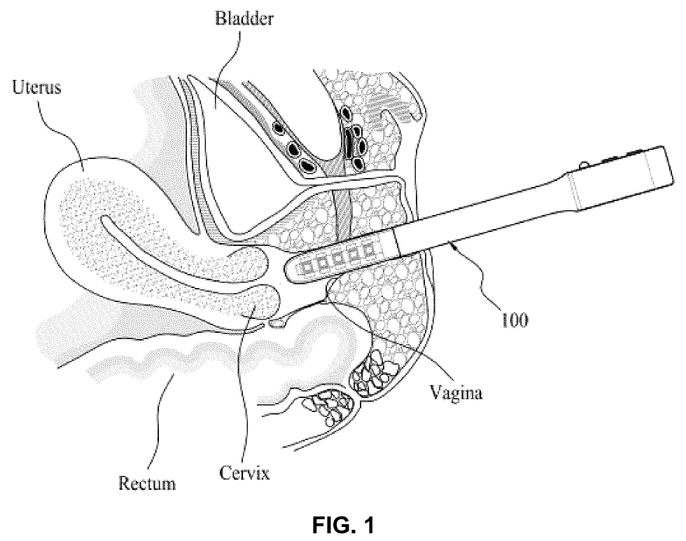

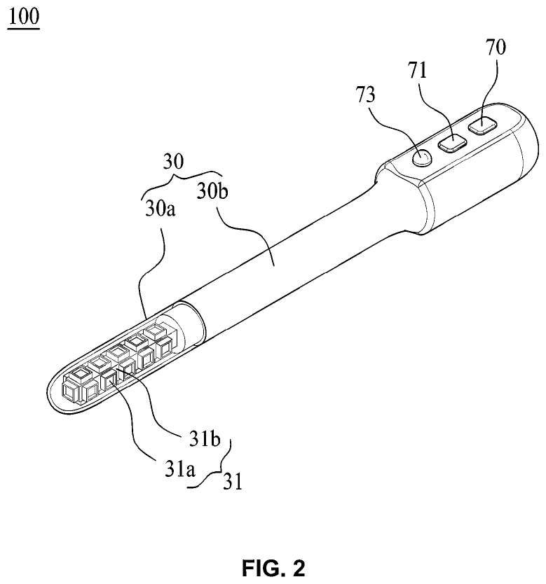

FIG. 1 is a schematic diagram of an illustrative apparatus inserted into vagina according to embodiments of the present disclosure, and FIG. 2 is a perspective diagram of an illustrative apparatus for inactivating viruses in a body according to embodiments of the present disclosure.

As depicted in FIG. 1 and FIG. 2, the apparatus 100 may comprise a body unit 30 with a light source that can be inserted into a human body (e.g., vagina) to irradiate light to vagina or cervix, and an operating unit 70 that can control the operation of the light source. In embodiments, the body unit 30 may include a light transmission part 30a that transmits light emitted from a light-emitting unit 31 placed therein to the outside, and a connecting part 30b that is coupled to the operating unit 70. The light-emitting unit 31 may be arranged with a plurality of Ultraviolet light emitting diodes (UV LEDs) packages 31a at regular intervals, and each UV LED package 31a operates according to a control signal from the operating unit 70.

In embodiments, the light transmission part 30a and connecting part 30b of the body unit 30 may be made of biocompatible materials that are harmless to the human body, and an outside surface of the body unit 30 may have a water-resistant property to allow cleaning. In particular, the connecting part 30b may be made of a flexible material that can be easily bent in the human body.

In embodiments, the UV LED 31a may be composed of at least two or more UV LED groups with different wavelengths, and for example, the UV LED 31a may be arranged in the light-emitting unit 31 by combining LEDs of various wavelengths that can irradiate ultraviolet rays in the UVA (Ultra Violet-type A: 315 nm˜400 nm), UVB (Ultra Violet-type B: 280 nm˜315 nm), and UVC (Ultra Violet-type C: 200 nm˜280 nm) regions.

In embodiments, the operating unit 70 may include a controller (not shown) formed therein side that is electrically connected to a button 71 for operating the apparatus and an indicator 73 that can transmit alert/notification signals for guiding the user. The operating unit 70 may also include a port (not shown) for charging or Internet communication. In addition, the operating unit 70 may further include a power button (not shown) for turning the power of the device on/off on one side. In embodiments, the operating unit 70 may control the brightness and wavelength of light emitted from the UV LED package 31a using the button 71, and the number of UV LED packages 31a that are operating. In addition, the operating unit 70 may control the emission pattern of the UV LED package depending on the number of times or length of the button 71 being pressed and control the mode setting or on/off for multiple emission patterns.

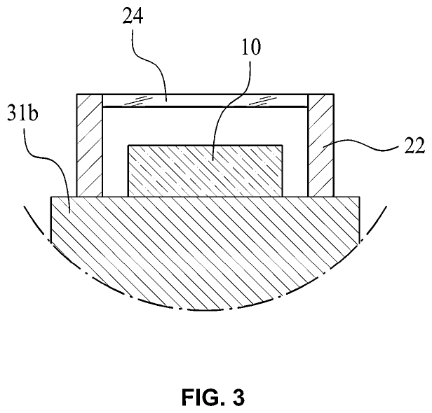

FIG. 3 is a cross-sectional diagram of a single UV LED package of a light-emitting unit according to embodiments of the present disclosure.

As depicted, the UV LED package 31a may include a UV LED chip 10 packaged in a housing 22. The UV LED chip 10 is mounted on a substrate 31b, which may be a metal or ceramic substrate with a circuit on it. The ceramic or metal housing 22 is firmly attached to the substrate 31b. A cap 24 may be completely sealed with the housing 22 and the UV light emitted from the UV LED chip 10 can pass through the cap 24. The cap 24 is for diffusing light, and the material of the cap may be quartz. The wavelength emitted by the UV LED chip 10 may be in several ranges, but it may be in the UVC range of 200-280 nm for inactivating HPV present in the cervix or vagina.

The radiation angle of the UV LED package 31a may be in the range of 120° to 140° if the cap 24 is flat, and in the range of 60° to 140° if the cap 24 is designed as an optical component. The radiation angle may be made large enough to cover the entire surface of the cervix by changing the structure and material of the cap, and multiple UV LED packages may be arranged to cover the entire surface of the cervix or vagina.

In embodiments, the light emitted from multiple UV LED packages 31a is sufficient to sterilize or inactivate various infectious organisms, including bacteria and HPV in the cervix or vagina. However, UV light, especially UVC light, may be dangerous if exposed to the skin for a long time. Therefore, the radiation angle is set to a specific limit below the specified limit by the Occupational Safety and Health Administration (OSHA) and the Environmental Protection Agency (EPA) in the United States so that UV light is safe when exposed to the skin.

The substrate 31b may be formed as a rectangular polyhedron with UV LED packages 31a arranged in a polygonal pattern, as depicted in FIG. 2, so that light can be irradiated not only to the cervix but also to a portion of the vagina. The circuit of the substrate 31b may be electrically connected to the controller of the operating unit 70.

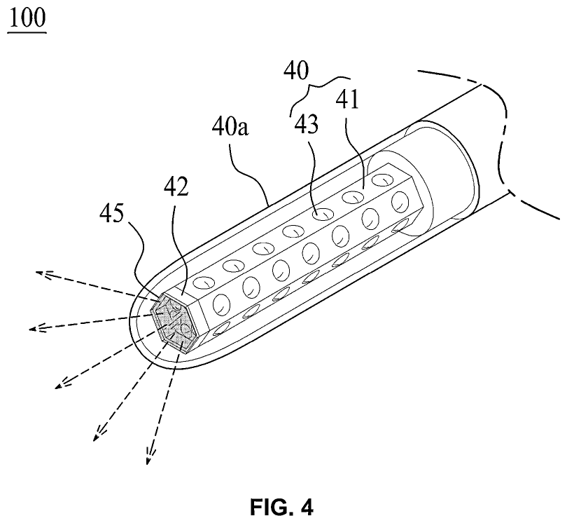

FIG. 4 is a diagram illustrating a portion of the body unit including a light-emitting unit according to a first embodiment of the present disclosure.

As depicted, a light-emitting unit 40 may include a rectangular polyhedral substrate 41, a plurality of UV LEDs 43 formed in chip type or package type on the substrate, and a diffuser 45 disposed on the upper surface of the substrate plane where the plurality of UV LEDs 43 is disposed facing the front of the apparatus 100.

The UV LEDs 43 may be arranged radially as shown but may be arranged in any form of arrangement that can widen the radiation angle. The diffuser 45 may be completely combined with the housing 42 formed along the edge of the front-facing substrate 41 surface, and UV light emitted from the UV LEDs 43 may pass through the diffuser 45.

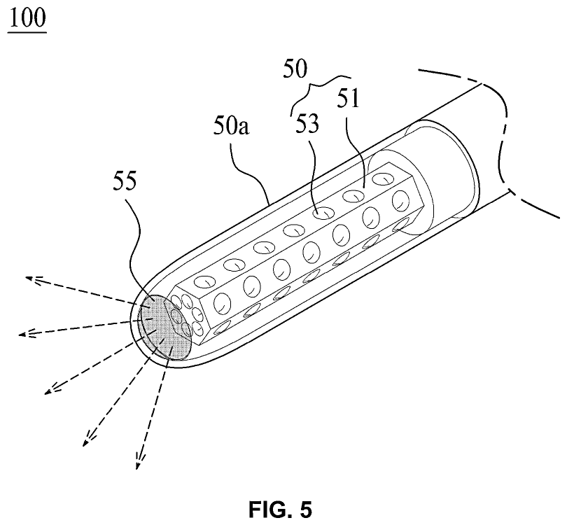

FIG. 5 is a diagram illustrating a portion of the body unit including a light-emitting unit according to a second embodiment of the present disclosure.

As depicted, a light-emitting unit 50 including a substrate 51 and a plurality of UV LEDs 53 may be formed and arranged similarly to the rectangular polyhedral substrate 41 and the plurality of UV LEDs 43 in FIG. 4. However, the diffuser 55 may be attached and arranged as a film over the entire surface of the light-transmitting portion 50a surrounding the light-emitting unit 50. For example, if the diffuser 55 is locally arranged on the light-transmitting portion 50a, it can be arranged on the light-transmitting portion 50a facing the UV LEDs located at the front of the apparatus 100.

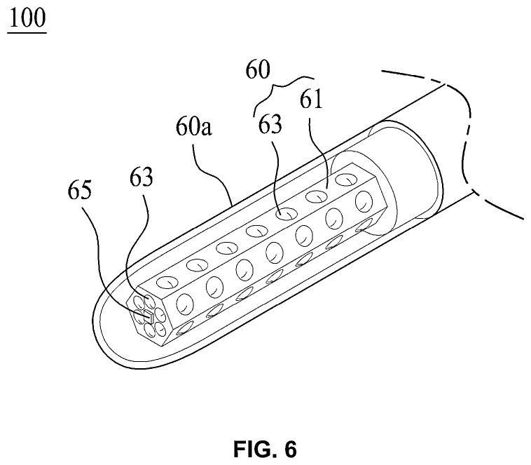

FIG. 6 is a diagram illustrating a portion of the body unit including a light-emitting unit according to a third embodiment of the present disclosure.

As depicted, a light-emitting unit 60 including a substrate 61 and UV LEDs 63 can be formed and arranged similarly to the rectangular polyhedral substrate 41 and the plurality of UV LEDs 43 in FIG. 4. Additionally, the diffuser (not shown) may be arranged on the upper surface of the substrate 61 or the light-transmitting portion 60a in the same way as depicted in FIG. 4 or 5.

An image sensor 65 may be arranged on the substrate 61 facing the front of the apparatus 100, along with the UV LEDs 63 located there. The image sensor 65 may be arranged in the center of the multiple UV LEDs arranged radially so that the apparatus 100 can image the vaginal environment and the cervix as it is inserted into the vagina and advances into the cervix.

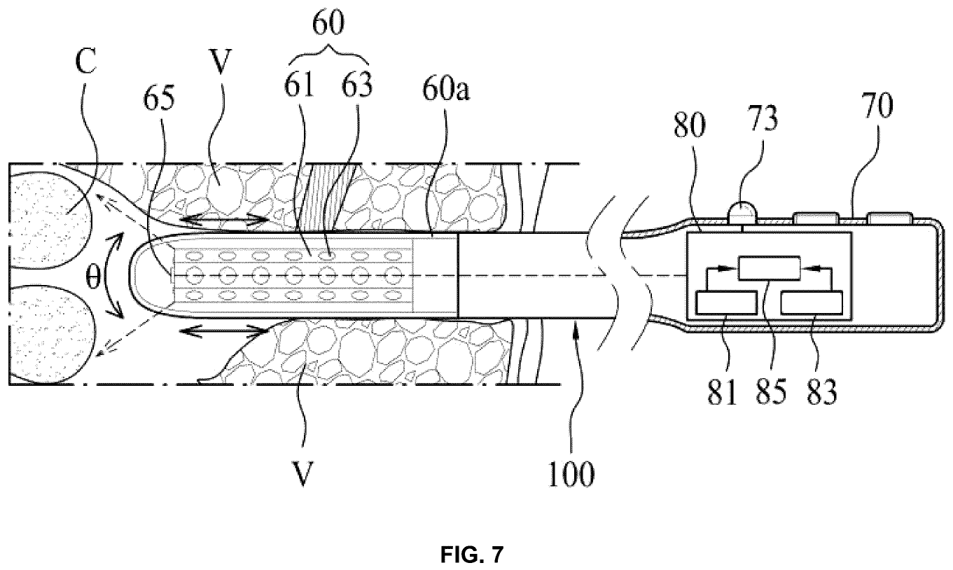

FIG. 7 is a diagram of an apparatus which includes a body unit in FIG. 6 according to embodiments of the present disclosure.

As depicted, the apparatus 100 may include UV LEDs 63 mounted on a substrate 61, an image sensor 65, and a controller 80. In one embodiment, the controller 80 may be mounted within the operating unit 70 of the apparatus 100 depicted in FIG. 2 and may be electrically connected to the indicator 73. The controller 80 may optically or electrically control the entire apparatus, and may include a measuring unit 81, a setting unit 83, and a computing unit 85.

The measuring unit 81 may measure the size of the cervix C as measured by the image sensor 65 included in the apparatus 100. The size of the cervix C changes as the body unit containing the image sensor 65 is inserted into the vagina V and approaches the cervix C.

The setting unit 83 may set the standard size of the cervix C when light is most efficiently irradiated to the cervix C based on the radiation angle (θ) of the light-emitting unit 60 facing the cervix C. These standard sizes may be stored in the setting unit 83 as a look-up table based on age, body type, and other medical information. In addition, the standard size may provide a location where light can be safely irradiated to the cervix C without causing physical damage to the cervix C when the light-emitting unit 60 is inserted into the vagina V.

The computing unit 85 may derive a result by comparing the measured cervical size as the light-emitting unit 60 moves through the vaginal canal into the cervix C with a pre-set standard size from the setting unit 83.

The controller 80 may transmit a signal to the indicator 73 based on the result obtained from the computing unit 85. For example, if the measured cervical size is smaller than the set standard size, the controller 80 may not transmit a signal to the indicator 73 to prevent any light from being emitted. This means that the light-emitting unit 60 is not yet in an efficient position to illuminate the cervix effectively. Accordingly, it allows the user to continue inserting the apparatus 100 further into the cervix direction.

Furthermore, if the measured cervical size matches the set standard size, the controller 80 may transmit a signal to the indicator 73 to be turned on. This means that a location of the light-emitting unit 60 for illuminating the cervix is efficient. Accordingly, it allows the user to stop inserting the apparatus 100 further into the cervix and allows UV light to be emitted at a stationary location of the light-emitting unit 60 by controlling the operating unit 70.

Additionally, if the measured cervical size is larger than the set standard size, the controller 80 may transmit a danger signal to the indicator 73 to be flicked. Because this means that the location of the apparatus 100 inserted into the vaginal is inefficient in irradiating light to the cervix, and if the apparatus is continuously inserted in a direction of the cervix, it may cause physical damage to the cervix. Accordingly, it allows the user to withdraw the apparatus 100 from the cervix C.

Thus, the apparatus 100 allows users to irradiate light safely and efficiently to the cervix without causing physical damage when inserting the apparatus 100 into the vagina, enabling the sterilization or inactivation of various infectious organisms, including the HPV, present in the vagina or cervix.

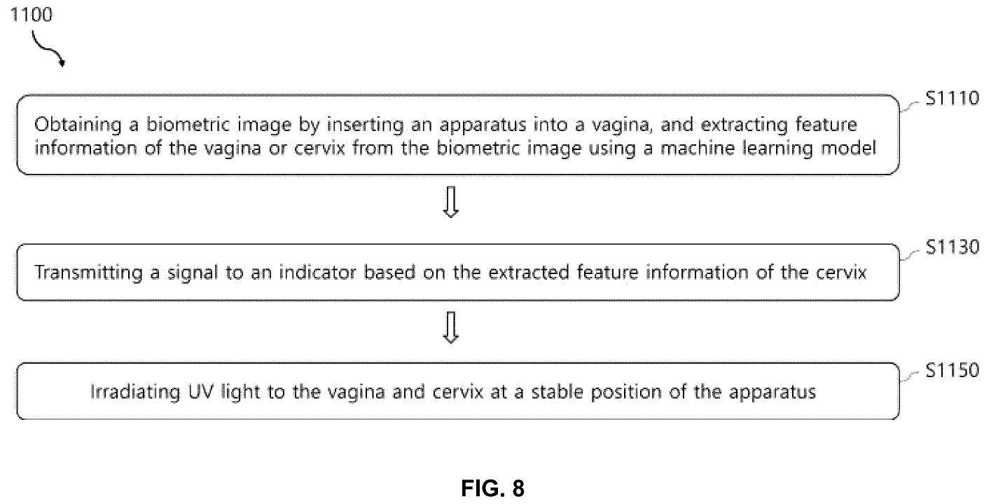

FIG. 8 is a flow diagram illustrating a method for inactivating viruses inside a human body according to an embodiment of the present disclosure. The method 800 may be performed by the apparatus 100 having UV light source.

At step S810, the apparatus 100 including an image sensor and UV LEDs is inserted into the vagina and advanced towards the cervix. The UV LEDs may encompass a wide range of wavelengths and remain inactive while in motion of the apparatus. At step S830, as the apparatus 100 approaches the cervix, a signal may be transmitted to the indicator based on the measured cervix size. That is, the signal, as an alert signal, is transmitted to the indicator 73 based on a change (e.g., increase) of the cervical size in a real-time. The cervix size may be measured by processing an image which is acquired by the image sensor 65 included into the apparatus 100. The alert signal may vary according to the angle of radiation of light or the distance from the light-emitting unit 40, 50, 60 to the cervix while the apparatus 100 irradiates UV light to the cervix. At step S850, when the apparatus 100 reaches a stable and efficient position to illuminate the cervix with the UV light based on the angle of radiation from the light-emitting unit 40, 50, 60 to the cervix, the apparatus 100 stops moving, and the UV LEDs is activated to illuminate the cervix or part of the vagina. The UV LEDs operation is controlled by the controller 80 with a built-in timer function and may be adjusted in frequency as needed.

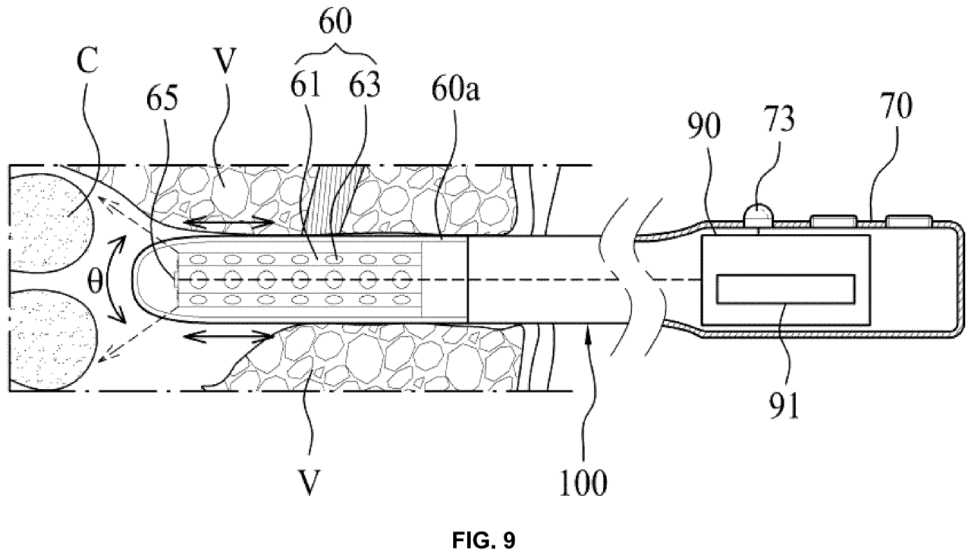

FIG. 9 is a diagram of another apparatus which includes a body unit in FIG. 6 according to embodiments of the present disclosure.

As depicted, the apparatus 100 may include a substrate 61 on which UV LEDs 63, an image sensor 65, and a controller 90 are arranged. In embodiments, the controller 90 may be placed inside the operating unit 70 of the apparatus 100 depicted in FIG. 2 and electrically connected to the indicator 73. The controller 90 may optically or electronically control the entire components of the apparatus 100. In embodiments, the controller 90 may include a memory 91 in where one or more machine learning models are stored. The controller 90 may perform the one or more machine learning models, which can be implemented in hardware, software, firmware, or a combination thereof. The controller 90 may be, but is not limited to, a processor, a processing module, a Computer Processing Unit (CPU), an Application Processor (AP), a microcontroller, a digital signal processor. In embodiments, the controller 90 may include an image filter such as a high pass filter or a low pass filter to filter a specific factor in cervix or virginal images. In addition, in embodiments, the controller 90 may communicate with a hardware controller such as a display adapter to display a user interface on a display device outside. In embodiments, the controller 90 may access the memory 91 and execute commands stored in the memory or one or more sequences of instructions to control the operation of the apparatus 100. The commands or sequences of instructions may be read in the memory from computer-readable medium or media such as a static storage or a disk drive but is not limited thereto. In alternative embodiments, a hard-wired circuitry which is equipped with a hardware in combination with software commands may be used. The hard-wired circuitry can replace the soft commands. The instructions may be an arbitrary medium for providing the commands to the controller 90 and may be loaded into the memory 91.

The machine learning model may be learned by data information, including first feature information (e.g., location, size, state) of the vagina or cervix images, or second feature information between the cervix and the angle of radiation of light emitted from the light-emitting unit 60. In embodiments, the image sensor 65 may capture images of the vagina and cervix in real time while moving to the cervix C. The controller 90 may extract feature information in real time from the captured images using a pre-learned machine learning model and control the indicator 73 based on the feature information. In embodiments, the feature information may be the size of the cervix obtained as the apparatus 100 is inserted into the vagina.

In embodiments, the controller 90 may generate signals for the operation of the indicator 73 based on the feature information. For example, if the feature information (e.g., the real-time measured cervical size) obtained from the machine learning model is smaller than the reference value, the controller 90 does not transmit any signal to the indicator 73 so that the indicator 73 is inactivated or turned off. Based on the operation of the indicator 73, it allows the user to continue inserting the light-emitting unit 60 into the cervix C. The reference value may be a standard size of the cervix C seen by the image sensor when light is most efficiently irradiated to the cervix based on the radiation angle.

In another case, if the feature information (e.g., the real-time measured cervical size) obtained from the machine learning model matches the reference value, the controller 90 does transmit a signal to the indicator 73 so that the indicator 73 is activated or turned on. Based on the operation of the indicator 73, it allows the user to stop further inserting the light-emitting unit 60 into the cervix C and the user may control the controller 70 to irradiate UV light to the cervix C in a position where the apparatus does not move. The reference value may be a standard size of the cervix seen by the image sensor when light is most efficiently irradiated to the cervix based on the radiation angle.

In further another case, if the feature information (e.g., the real-time measured cervical size) obtained from the machine learning model is larger than the reference value, the controller 90 does transmit a specific signal to the indicator 73 so that the indicator 73 is flicked. The specific signal is for informing the user of danger information. Because the position of the apparatus 100 inserted into the vagina is inefficient in irradiating light to the cervix C, and the apparatus 100 is continuously inserted in the direction of the cervix, a physical damage may be caused to the cervix C. Based on the operation of the indicator, it allows the user to withdraw the apparatus 100 from the vagina.

As such, the apparatus 100 enables the users to irradiate light safely and efficiently to the cervix without causing physical damage when inserting the apparatus 100 into the vagina, thereby allowing for the sterilization or inactivation of various infectious organisms, including the HPV present in the vagina or cervix.

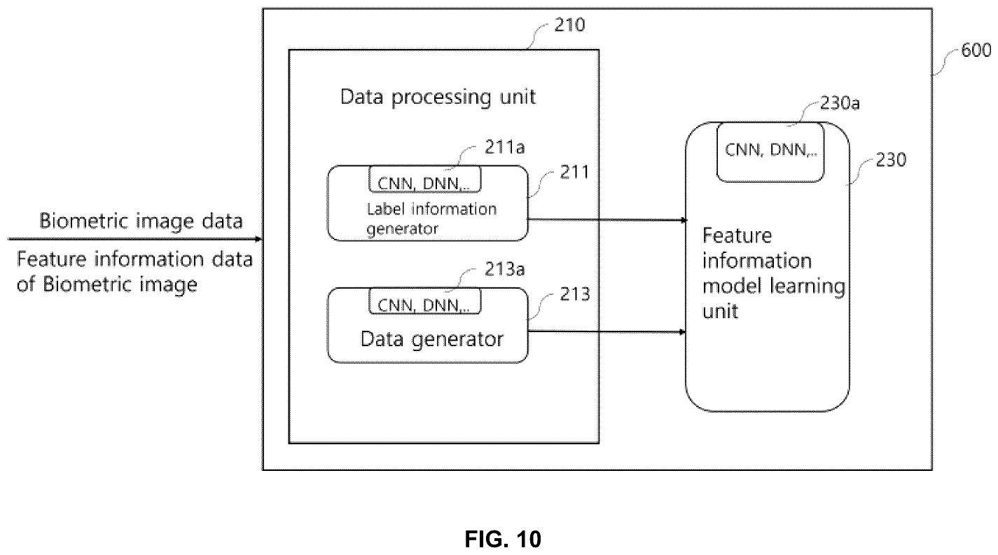

FIG. 10 is a block diagram explaining a training method of a controller of an apparatus according to embodiments of the present disclosure.

As depicted, the controller 600 receives training data to train the machine learning model (211a, 213a, 215a, 230a) and may extract feature information from the received training data based on the machine learning model. The training data may be a real-time biometric image data obtained through the image sensor or feature information data extracted from real-time biometric image data. In one embodiment, the feature information extracted from the real-time biometric image data may include label information that classifies targets recognized in the biometric image data, such as organs within the body like the vagina or cervix. The label information may include the position information (e.g., 2D or 3D coordinates) of the target, the size information (e.g., width and height) of the target. The label may be given weight or order based on a biometric meaning of the target recognized in real-time biometric image data.

In embodiments, the controller 600 may include a data processing unit 210 and a feature information model learning unit 230. The data processing unit 210 may receive real-time biometric image data and feature information data of the real-time biometric image data for training the feature information model learning unit 230 and may transform or image process the received biometric image data and feature information data of the biometric image into suitable data for training of a feature information model. The data processing unit 210 may include a label information generator 211 and a data generator 213.

The label information generator 211 may generate label information corresponding to the received real-time biometric image data using a first machine learning model 211a. The label information may represent information about one or more categories based on the target which is recognized within the received real-time biometric image data. In one embodiment, the label information may be stored in memory along with information corresponding to the real-time biometric image data.

The data generator 213 may generate data to be input to the feature information model learning unit 230 that includes the machine learning model 230a. The data generator 213 may use a second machine learning model 213a to generate input data for a third machine learning model 230a based on multiple frame data included in the received real-time biometric image data. Frame data may represent each frame constituting real-time biometric images, and it may include RGB data for each frame, or data extracted from the characteristics of each frame or represented as vectors for each frame.

The feature information model learning unit 230 may include the third machine learning model 230a. The third machine learning model 230a may extract feature information about the real-time biometric image data by fusion learning the data including the label information and image data which are generated and extracted from the label information generation unit 211 and the data generation unit 213, respectively.

Feature information may be information related to a characteristic of a target image recognized from the real-time biometric image data. For example, the feature information may be a label such as cervix information that classifies an object within biometric image data. If an error occurs in the feature information extracted from the feature information model learning unit 230, the coefficients or connection weight values used in the third machine learning model 230a may be updated.

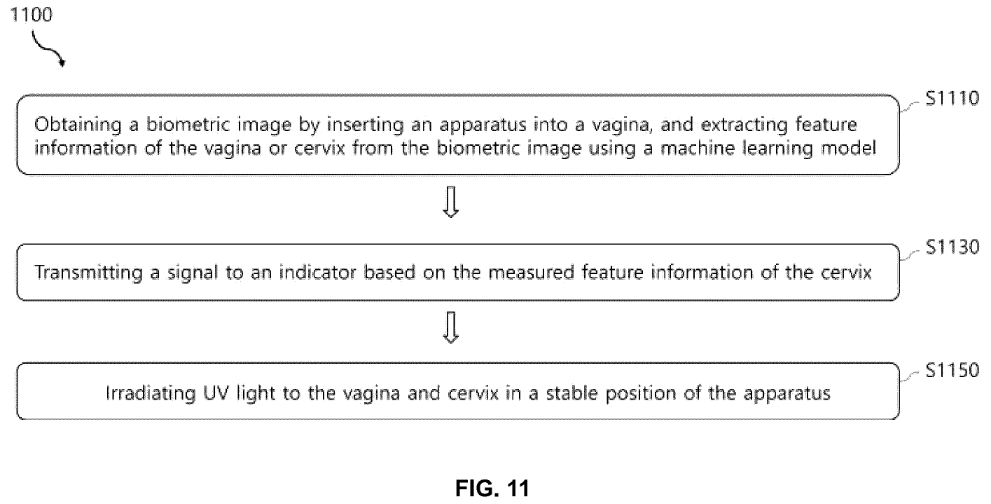

FIG. 11 is a flow diagram illustrating a method for inactivating viruses inside a human body according to another embodiment of the present disclosure. The method 1100 may be performed by the apparatus 100 having UV light source.

In the apparatus 100, the machine learning model may be utilized to extract feature information from real-time captured biometric images. The feature information may be information that can be labeled into a category such as the vagina or cervix of the human body within a biometric image. The machine learning model may include, be not limited into, deep neural networks (DNN), convolutional neural networks (CNN), recurrent neural networks (RNN), or other machine learning algorithms.

At step S1110, biometric images captured in real time by inserting the apparatus 100 into the vagina is input to a machine learning model, and feature information of the vagina or cervix may be extracted from the input biometric images based on the machine learning model. In embodiments, the captured biometric images may be dynamic video images of tissues inside the body. The feature information of the cervix may include at least one of the presence, size, and of the cervix. The location of the cervix can be represented by 2D or 3D coordinates.

At step S1130, based on the extracted feature information of the cervix, the controller 80, 90 may transmit a signal to the indicator 73 to indicate whether the apparatus 100 is moving. At this time, the signal may be transmitted to the indicator 73 in various forms depending on the location of the cervix the range of the radiation angle of light emitted from the light emitting unit 60 compared to the size of the cervix.

At step S1150, the apparatus 100 may irradiate UV light to the vagina and cervix at a stable position of the apparatus. That is, while the apparatus 100 is moving to the cervix, when the radiation angle of the light reaches a position where the UV light can be safely and efficiently irradiated to the cervix according to the distance from the light emitting unit 60 to the cervix, the apparatus 100 stops moving and operates the UV LEDs is turned on. Afterwards, UV light is irradiated to the cervix or part of the vagina. At this time, the UV LEDs operates only for a certain period under the control of the controller 80, 90 with a built-in timer function, and the number of times may be adjusted as needed.

Embodiments of the present disclosure may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

In the description, numerous details are set forth for purposes of explanation to provide a thorough understanding of the present disclosure. However, it will be apparent to one skilled in the art that not all these specific details are required in order to practice the present disclosure.

Additionally, while specific embodiments have been illustrated and described in this specification, those of ordinary skill in the art appreciate that any arrangement that is calculated to achieve the same purpose may be substituted for the specific embodiments disclosed. This disclosure is intended to cover any and all adaptations or variations of the present invention, and it is to be understood that the terms used in the following claims should not be construed to limit the invention to the specific embodiments disclosed in the specification. Rather, the scope of the invention is to be determined entirely by the following claims, which are to be construed in accordance with the established doctrines of claim interpretation, along with the full range of equivalents to which such claims are entitled.

What is claimed is:

1. An apparatus for inactivating viruses in human body, comprising:
a body unit including a light emitting unit and formed to be capable of inserting into the human body;
an operating unit coupled to the body unit to control the light emitting unit; and
a diffusor disposed on an upper portion of the light emitting unit to diffuse light emitted from the light emitting unit,
wherein the light emitting unit includes a substrate having a rectangular polyhedral shape and a plurality of ultraviolet light emitting diodes (LEDs) disposed on a surface of the substrate.

2. The apparatus of claim 1, wherein the diffusor is coupled with a housing formed along an edge of the substrate.

3. The apparatus of claim 2, wherein the diffusor is disposed in a film form on a light transmission part of the body unit.

4. The apparatus of claim 1, wherein the light emitting unit further includes an image sensor disposed on the substrate, and the image sensor is disposed at a center of the substrate.

5. The apparatus of claim 4, wherein the operating unit includes an indicator that displays a signal for an user guidance based on information obtained by the image sensor.

6. The apparatus of claim 1, wherein the operating part includes a button that is electrically connected to the light emitting and controls the light emitting unit, and the button controls at least one of an illuminance of light, a wavelength of light, an emission pattern of light emitted from the plurality of ultraviolet LEDs.

7. The apparatus of claim 1, wherein the plurality of ultraviolet LEDs includes at least two groups of ultraviolet LEDs with different wavelengths.

8. An apparatus for inactivating viruses in human body, comprising:
a body unit that includes a light emitting unit and formed to be capable of inserting into the human body; and
an operating unit that is coupled to the body unit and includes a controller for electrically controlling the light emitting unit,
wherein the light emitting unit includes a substrate, each of an image sensor and a plurality of ultraviolet light emitting diodes (LEDs) disposed on the substrate, and
wherein the light emitting unit irradiates ultraviolet light to a cervix based on a cervix image captured by the image sensor.

9. The apparatus of claim 8, wherein the image sensor is disposed on a center of the substrate, the plurality of the ultraviolet LEDs are radially arranged around the image sensor.

10. The apparatus of claim 8, wherein the controller includes a machine learning model and extracts feature information from the cervix image using the machine learning model, and generates a signal for operating an indicator of the operating unit according to the feature information.

11. The apparatus of claim 10, wherein the feature information includes at least one of a presence, a size, a location of the cervix.

12. The apparatus of claim 8, wherein the controller generates a signal for operating an indicator of the operating unit according to a size of the cervix obtained from the cervix image.

13. The apparatus of claim 8, wherein the controller generates a signal for operating an indicator of the operating unit by comparing a size of the cervix obtained from the cervix image with a reference size of the cervix.

14. The apparatus of claim 13, wherein the reference size of the cervix is set differently depending on age.

15. An apparatus for inactivating viruses in human body, comprising:
an image sensor that is inserted into a vagina to take an image of a cervix;
a controller that extracts feature information of the image of the cervix using a machine learning model; and
a light source that irradiates ultraviolet light to the vagina or the cervix based on the feature information.

16. The apparatus of claim 15, further comprising:
an indicator operated under control of the controller according to a reference value of the feature information.

17. The apparatus of claim 15, wherein the feature information includes at least one of a presence, a size, a location of the cervix.
